(19) United States
(12) Patent Application Publication
(10) Pub. No.: US 2008/0200387 A1
(43) Pub. Date: Aug. 21, 2008

(54) ANTI-ANGIOGENIC PROTEIN, COMPOSITION AND USE THEREOF

(76) Inventors: Hua-Lin Wu, Tainan (TW); Guey-Yueh Shi, Tainan (TW)

Correspondence Address: WPAT, PC;INTELLECTUAL PROPERTY ATTORNEYS, 2030 MAIN STREET, SUITE 1300, IRVINE, CA, 92614, US

(21) Appl. No.: 12/030,847

(22) Filed: Feb. 13, 2008

Related U.S. Application Data
(60) Provisional application No. 60/867,243, filed on Feb. 15, 2007.

(51) Int. Cl. A61K 38/16; C07K 14/00; A61K 31/711; A61P 7/00; A61P 35/04; C07H 21/04

(52) U.S. Cl. 514/12; 530/350; 536/23.1; 514/44

(57) ABSTRACT

The present invention relates to an anti-angiogenic protein comprising a mutant kringle 1-5 (K₁₋₅) fragment of plasminogen, wherein a set of mutation position is selected from the group consisting of positions 227, 284 and 470 of SEQ ID NO. 6, and wherein the position 227 is replaced with an amino acid residue without forming glycosylation, the position 284 is replaced with an amino acid residue without forming glycosylation, and the position 470 is replaced with Arg. The present invention also provides a nucleic acid having a sequence encoding said anti-angiogenic protein. The invention further relates to a pharmaceutical composition for inhibiting angiogenesis comprising said anti-angiogenic protein or said nucleic acid. The present invention also provides a method for treatment of an angiogenesis associated disease or disorder, comprising administering to a patient in need of such treatment an effective amount of said pharmaceutical composition.

### FIELD OF THE INVENTION

The present invention relates to an anti-angiogenic protein and a nucleic acid having a sequence encoding said protein. The invention further relates to a pharmaceutical composition for inhibiting angiogenesis. Also disclosed is a method for treatment of an angiogenesis associated disease or disorder.

### BACKGROUND OF THE INVENTION

Angiogenesis plays an essential role in several physiological processes such as wound healing, female reproduction, embryogenic development, organ formation, and tissue regeneration and remodeling (Folkman J, Shing Y Angiogenesis. J Biol Chem 1992; 267: 10931-10934; Folkman J. Seminars in Medicine of the Beth Israel Hospital, Boston. Clinical applications of research on angiogenesis. N Engl J Med 1995; 333: 1757-1763). Several steps are needed to develop new blood vessels, including the need for original quiescent endothelial cells to degrade the local basement membrane, to change cell morphology, to proliferate, to invade surrounding stromal tissues, to sprout new capillary branches, and to reconstitute new basement membrane (Folkman J, Shing Y Angiogenesis. J Biol Chem 1992; 267: 10931-10934). The complex angiogenic processes that can be transiently switched on or off are under the control of angiogenesis enhancers and inhibitors (Folkman J. Seminars in Medicine of the Beth Israel Hospital, Boston. Clinical applications of research on angiogenesis. N Engl J Med 1995; 333: 1757-1763; O'Reilly M S, Holmgren L, Shing Y, et al. Angiostatin: a novel angiogenesis inhibitor that mediates the suppression of metastases by a Lewis lung carcinoma. Cell 1994; 79: 315-328). Under pathological conditions, abnormal growth of new blood vessels can lead to the progression of many diseases, including diabetic retinopathy and tumor growth (Folkman J. Seminars in Medicine of the Beth Israel Hospital, Boston. Clinical applications of research on angiogenesis. N Engl J Med 1995; 333: 1757-1763; Cao Y, Ji R W, Davidson D, et al. Kringle domains of human angiostatin. Characterization of the anti-proliferative activity on endothelial cells. J Biol Chem 1996; 15: 29461-29467).

Several studies have provided direct and indirect evidence that tumor growth and metastasis are angiogenesis-dependent (O'Reilly M S, Holmgren L, Shing Y, et al. Angiostatin: a novel angiogenesis inhibitor that mediates the suppression of metastases by a Lewis lung carcinoma. Cell 1994; 79: 315-328; Folkman J. Angiogenesis in cancer, vascular, rheumatoid and other disease. Nat Med 1995; 1: 27-31). Such evidence indicates, therefore, that blocking excessive angiogenesis in cancer is a promising therapeutic strategy. Numerous endogenous angiogenic inhibitors have been identified, and several of them are currently being investigated in clinical trials for cancer therapies. One of these inhibitors, angiostatin, a proteolytic fragment of plasminogen, was isolated in 1994 (O'Reilly M S, Holmgren L, Shing Y, et al. Angiostatin: a novel angiogenesis inhibitor that mediates the suppression of metastases by a Lewis lung carcinoma. Cell 1994; 79: 315-328). When administered systemically, angiostatin significantly inhibits primary tumor growth, angiogenesis-dependent growth of metastases, and corneal neovascularization in mice (O'Reilly M S, Holmgren L, Chen C, et al. Angiostatin induces and sustains dormancy of human primary tumors in mice. Nat Med 1996; 2: 689-692; Ji W R, Castellino F J, Chang Y, et al. Characterization of kringle domains of angiostatin as antagonists of endothelial cell migration, an important process in angiogenesis. FASEB J 1998; 12: 1731-1738; Cao R, Wu H L, Veitonmaki N, et al. Suppression of angiogenesis and tumor growth by the inhibitor K1-5 generated by plasmin-mediated proteolysis. Proc Natl Acad Sci USA 1999; 96: 5728-5733). These anti-tumor effects correlate with a marked reduction of microvessel density within the tumor mass, indicating that suppression of angiogenesis may be associated with inhibiting tumor growth (Cao R, Wu H L, Veitonmaki N, et al. Suppression of angiogenesis and tumor growth by the inhibitor K1-5 generated by plasmin-mediated proteolysis. Proc Natl Acad Sci USA 1999; 96: 5728-5733). In addition, other small kringle fragments of human angiostatin from different expressed systems also exhibit different inhibitory activity on endothelial cell proliferation and migration in vitro. Kringle 1-3 has been shown to have more potent anti-endothelial cell proliferative activity than kringle 1-4 (angiostatin), suggesting that the kringle 4 structure may prevent some of the inhibitory activity. Although kringle 4 has marginal anti-proliferative activity, recent studies showed that kringle 4 is the most potent fragment in inhibiting endothelial cell migration and that the combination of kringle 1-3 and kringle 4 resulted in anti-migratory activity comparable to that of angiostatin. Therefore, investigators suggested that different kringle domains might contribute to the overall anti-angiogenic function of angiostatin by their distinct activities. Additionally, a synergistic inhibitory effect on endothelial cell proliferation from the co-incubation of angiostatin and kringle 5 was shown to be comparable to that of kringle 1-5 (also abbreviated as K₁₋₅ or K1-5) and to be greater than that of angiostatin. The mechanism of converting plasminogen to angiostatin in vivo is not clear. Some reports show that activated human neutrophils, macrophages, and tumor cells may generate angiostatin-like fragments.

Human plasminogen exists in two major molecular glycoforms, type 1 (˜33%) and type 2 (˜67%) in circulation (Pirie-Shepherd S R. Role of carbohydrate on angiostatin in the treatment of cancer. J Lab Clin Med 1999; 134: 553-560). The two types differ only in their carbohydrate content (Hayes M L, Castellino J E Carbohydrate of the human plasminogen variants. I. Carbohydrate composition, glycopeptide isolation, and characterization. J Biol Chem 1979; 254: 8768-8771; Davidson D J, Castellino F J. Oligosaccharide structures present on asparagine-289 of recombinant human plasminogen expressed in a Chinese hamster ovary cell line. Biochemistry 1991; 30: 625-633). Type 1 glycoform of plasminogen contains one N-linked glycosylation at residue Asn-289 and one O-linked glycosylation at residue Thr-346. Type 2 glycoform of plasminogen exists in only O-linked glycosylation at Ser-248 and Thr-346 (Davidson D J, Castellino F J. Oligosaccharide structures present on asparagine-289 of recombinant human plasminogen expressed in a Chinese hamster ovary cell line. Biochemistry 1991; 30: 625-633; Pirie-Shepherd S R, Stevens R D, Andon N L, et al. Evidence for a novel O-linked sialylated trisaccharide on Ser-248 of human plasminogen 2. J Biol Chem 1997; 272: 7408-7411). Moreover, angiostatin derived from a different glycoform of plasminogen was shown to exhibit different anti-proliferation activity (Pirie-Shepherd S R. Role of carbohydrate on angiostatin in the treatment of cancer. J Lab Clin Med 1999; 134: 553-560). This implies that glycosylation may be involved in the anti-angiogenic effect. Carbohydrate chains may also influence protein folding, clearance rate, protease resistance, and even the intrinsic functions of proteins, such as the receptor binding ability. In addition, the existence of oligosaccharide chains may mask the recognition of surface areas in protein interaction (Pirie-Shepherd S R. Role of carbohydrate on angiostatin in the treatment of cancer. J Lab Clin Med 1999; 134: 553-560).

Structures of four of the five individual plasminogen kringle domains and kringle 1-3 have been determined crystallographically. Their binding modes for Lys-like ligands have been extensively studied both structurally and by site-directed mutagenesis (Chang Y, Mochalkin I, McCance S G, et al. Structure and ligand binding determinants of the recombinant kringle 5 domain of human plasminogen. Biochemistry 1998; 37: 3258-3271; McCance S G, Menhart N, Castellino F J. Amino acid residues of the kringle-4 and kringle-5 domains of human plasminogen that stabilize their interactions with omega-amino acid ligands. J Bio). Kringle 1, kringle 4, and kringle 5 have relatively high affinity for 6-aminocaproic acid (EACA), a Lys residue mimic, while kringle 2 has relatively weak binding affinity. In contrast, there is no binding for EACA in kringle 3 (Marti D, Schaller J, Ochensberger B, et al. Expression, purification and characterization of the recombinant kringle 2 and kringle 3 domains of human plasminogen and analysis of their binding affinity for omega-aminocarboxylic acids. Eur J Biochem 1994; 219: 455-462). Replacement of Leu-71 (corresponding to residue 532 in K1-5), the Lys binding site on kringle 5, by Arg increases its Lys binding affinity (Chang Y, Mochalkin I, McCance S G, et al. Structure and ligand binding determinants of the recombinant kringle 5 domain of human plasminogen. Biochemistry 1998; 37: 3258-3271). Previous studies of individual kringle domains other than kringle 5 show that the Lys binding property may have no correlation with the anti-angiogenesis ability of kringle domains (Cao Y. Ji R W, Davidson D, et al. Kringle domains of human angiostatin. Characterization of the anti-proliferative activity on endothelial cells. J Biol Chem 1996; 15: 29461-29467). Nevertheless, the role of the Lys binding property of K₁₅ in inhibiting angiogenesis remains unclear.

The anti-angiogenesis mechanism of angiostatin or K₁₋₅ is currently under investigation. Some molecules were reported as angiostatin-related protein receptors, including ATP synthase, integrin αᵥβ₃, and angiomotin. It is reported that angiostatin-related proteins bind to ATP synthase on the cell surface and the binding of angiostatin to ATP synthase may mediate the anti-angiogenic effects of angiostatin, and then down-regulate endothelial cell proliferation, migration, and apoptosis. Moreover, integrin αᵥβ₃ may be a predominant receptor for angiostatin on BAECs. In 2001, angiomotin, a protein that mediates angiostatin inhibition of migration and tube formation of endothelial cells, was identified using the yeast two-hybrid system. The physiological relationship of these potential receptors remains to be elucidated.

### SUMMARY OF THE INVENTION

The present invention relates to an anti-angiogenic protein comprising a mutant kringle 1-5 (K₁₅) fragment of plasminogen, wherein the mutation has substitution of amino acid residue of SEQ ID No. 6 at amino acid position selected from the group consisting of 227, 284 and 470, and wherein the position 227 is replaced with an amino acid residue without forming glycosylation, position 284 is replaced with an amino acid residue without forming glycosylation, and the position 470 is replaced with Arg or Leu; provided that the position 227 being Asn, position 284 being Thr and position 470 being Leu is excluded.

The invention also relates to a nucleic acid having a sequence encoding said anti-angiogenic protein of the invention.

The invention further relates to a pharmaceutical composition for inhibiting angiogenesis comprising said anti-angiogenic protein or nucleic acid of the invention. Also disclosed is a method for treatment of an angiogenesis associated disease or disorder, comprising administering to a patient in need of such treatment an effective amount of said pharmaceutical composition.

### BRIEF DESCRIPTION OF THE DRAWINGS

FIG. 1 shows the analysis of wild-type and mutant K₁₋₅ (kringle 1-5) by gel electrophoresis. SDS-PAGE was performed under reducing condition and proteins were detected by Coomassie blue staining. Lane 1: wild-type K₁₋₅, Lane 2: K₁₋₅N289A, Lane 3: K₁₋₅T346A, Lane 4: K₁₋₅L532R, Lane 5: K₁₋₅N289A/T346A, Lane 6: K₁₋₅T346A/L532R, Lane 7: K₁₋₅N289A/L532R, Lane 8: K₁₋₅N289A/T346A/L532R.

FIG. 2 shows inhibition of endothelial cell proliferation by K₁₋₅ proteins. The anti-proliferative effect of K₁₋₅ proteins on BAECs (A) and HMECs (B) was assayed in the presence of 10 ng/ml bFGE The inhibitory activity of K₁₋₅ proteins was shown by absorbance (means ±SD, n=4). *p<0.05; **p<0.01; ***p<0.001 versus bFGF-treated control, and ^{#}p<0.05; ^{##}p<0.01; ^{###}p<0.001 versus wild-type K₁₋₅.

FIG. 3 shows endothelial cell apoptosis detection. BAECs (A) and HMECs (B) were incubated in the presence of 200 nM K₁₋₅ proteins with 10 ng/ml bFGF for 24 h. Data are shown as the means of the ratio of sub G₁ to indicate apoptotic cells (means SD, n=3). ***p<0.001 versus bFGF-treated control, and ^{##}p<0.01; ^{###}p<0.001 versus wild-type K₁₋₅.

FIG. 4 shows in vivo angiogenesis assay. C57BL6/J mice were each injected subcutaneously with 0.5 ml Matrigel containing bFGF and in the presence or absence of K₁₅ proteins (wild-type K₁₋₅, K₁₋₅N289A, K₁₋₅T346A, K₁₋₅L532R, or K₁₋₅N289A/T346A/L532R) with heparin (60 U/ml) near the abdominal midline. After 7 days, mice were sacrificed and Matrigel plugs were excised and photographed. Angiogenesis induced by bFGF was compared with negative control (PBS). Sections of the recovered gels were immunofluorescence-stained for the presence of CD31. Results are representative of three separate experiments. Appearance of Matrigel plugs recovered on day 7 induced by bFGF in the presence and absence of kringle proteins. (A) PBS or bFGF only, (B) bFGF plus 80 or 160 nM wild-type or mutant K₁₋₅ proteins, (C) The hemoglobin content of Matrigel plugs was calculated as an indicator of angiogenesis. p<0.001 versus bFGF-treated control, and ^{#}p<0.05 versus K₁₋₅ 160 nM treatment.

FIG. 5 shows suppression of primary tumor growth by systemic administration of K₁₋₅ proteins. Lewis lung carcinoma cells (1×10⁶ cells) were implanted subcutaneously in the middle dorsum of C57BL6/J mice. Systemic treatment by subcutaneous injections once per two days with either 100 μl of PBS, kringle proteins (wild-type K₁₋₅ or K₁₋₅N289A/T346A/L532R) 2.5 mg/kg/day in PBS was started on the 7^{th} day after tumor implantation (n=12/each group) continuing through day 21. (A) Representative graphs of tumor mass of Lewis lung carcinoma bearing mice treated with PBS, wild-type K₁₋₅, or K₁₋₅N289A/T346A/L532R at day 21 after treatment. (B) Tumor volumes of control PBS-treated group (), wild-type K₁₋₅-treated group (▴), and K₁₋₅N289A/T346A/L532R-treated group (▪) were calculated by the formula: width²×length×0.52. Data represent the tumor volume (mean ±SD) of 12 mice in each group. ***p<0.001 versus PBS-treated control, and #p<0.05 versus wild-type K₁₋₅ treatment. (C) Immunohistochemical analysis of neovascularization of primary tumors treated with PBS, wild-type K₁₋₅, or K₁₋₅N289A/T346A/L532R; and tumors recovered at day 21. Tumor histological sections were stained with an anti-CD31 antibody and quantified by image pro-plus software. ***p<0.001 versus PBS-treated control, and ^{###}p<0.001 versus wild-type K₁₋₅.

FIG. 6 shows the ability of endothelial cells bound to K₁₋₅ and K₁₋₅N289A/T346A/L532R. Adhesion of BAECs (A) and HMECs (B) to BSA-, K₁₋₅- and K₁₋₅N289A/T346A/L532R-coated wells was determined by endogenous phosphatase activity as described in methods. The amounts of these wells coated with K₁₋₅ or K₁₋₅ N289A/T346A/L532R were equal according to ELISA assay analysis. Values represent means ±SD, n=4. **p<0.01; ***p<0.001 versus BSA-coated control and ^{#}p<0.05; ^{##}p<0.001 versus K₁₋₅ treatment. BAECs (C) and HMECs (D) were pretreated with 10 μg/ml anti-integrin αᵥβ₃ (Ab1), anti-integrin α₂β₁ (Ab2) antibodies, or normal mouse IgG (Ab3) for 30 min before seeding to protein-coated 96-well plate, and then detected. Values represent means ±SD, n=4. ***p<0.001 versus respective K₁₋₅ and K₁₋₅N289A/T346A/L532R treatment. (E and F) The binding ability of biotin-labeled kringle proteins to BAECs was assayed by ELISA as described in methods. (E) Values represent means ±SD, n=4. *p<0.05; **p<0.01; ***p<0.001 versus BSA-coated control, and ^{#}p<0.05; ^{##}p<0.01 versus K₁₋₅ treatment. (F) Values represent means ±SD, n=4. ***p<0.001 versus respective K₁₋₅ and K₁₋₅N289A/T346A/L532R treatment. (G) Adhesion of HMECs to 62.5 nM BSA-, K₁₋₅-, K₁₋₅N289A/T346A/L532R—, vitronectin- and fibronectin-coated wells was determined (means ±SD, n=3). p<0.001 versus BSA-coated control and ###p<0.001 versus K₁₋₅ treatment.

FIG. 7 shows the inhibitory effect of kringle proteins on endothelial cell proliferation mediated through integrin αᵥβ₃. HMECs were pretreated with 10 μg/ml anti-integrin αᵥβ₃ (Ab1) and anti-integrin α₂β₁ (Ab2) antibodies for 30 min and then treated with kringle proteins for 3 days. The result was analyzed by BrdU incorporation assay (means ±SD, n=3). ***p<0.001 versus bFGF-treated control, and ^{##}p<0.01; ^{###}p<0.001 versus respective kringle proteins.

### DETAILED DESCRIPTION OF THE INVENTION

Many proteins contain kringle structure, including apolipoprotein, prothrombin, tissue-type plasminogen activator. Recently, several studies indicated that these recombinant kringle domains demonstrated differential effects in anti-angiogenesis ability (Cao Y, Ji R W, Davidson D, et al. Kringle domains of human angiostatin. Characterization of the anti-proliferative activity on endothelial cells. J Biol Chem 1996; 15: 29461-29467; Cao Y, O'Reilly M S, Marshall B, et al. Expression of angiostatin cDNA in a murine fibrosarcoma suppresses primary tumor growth and produces long-term dormancy of metastases. J Clin Invest 1998; 101: 1055-1063; Chen Y H, Wu H L, Li C, et al. Anti-angiogenesis mediated by angiostatin K1-3, K1-4 and K1-4.5. Involvement of p53, FasL, AKT and mRNA deregulation. Thromb Haemost 2006; 95: 668-77). Results of these studies suggest that kringle structure may play a specific role in mediating endothelial-dependent anti-angiogenesis. Glycosylation of protein may mask the recognition site on the protein surface (Pirie-Shepherd S R. Role of carbohydrate on angiostatin in the treatment of cancer. J Lab Clin Med 1999; 134: 553-560), and decrease the chance of protein-protein interaction. In a preferred embodiment of the invention, the glycosylation site at Asn-289 and Thr-346 were eliminated and the effect on anti-angiogenic potency was determined. Previous results of anti-angiogenic effects of individual kringle domains within kringle 1-4 indicated that Lys binding feature has no correlation with the anti-angiogenesis effects (Cao Y, Ji R W, Davidson D, et al. Kringle domains of human angiostatin. Characterization of the anti-proliferative activity on endothelial cells. J Biol Chem 1996; 15: 29461-29467). Among several angiogenesis inhibitors, K₁₋₅ and kringle 5 were more potent in inhibiting endothelial cell proliferation and migration (Cao R, Wu H L, Veitonmaki N, et al. Suppression of angiogenesis and tumor growth by the inhibitor K1-5 generated by plasmin-mediated proteolysis. Proc Natl Acad Sci USA 1999; 96: 5728-5733; Dong Z, Kumar R, Yang X, et al. Macrophage-derived metalloelastase is responsible for the generation of angiostatin in Lewis lung carcinoma. Cell 1997; 88: 801-810). It appears that kringle 5 domain of plasminogen may play a unique role compared with other kringle domains in anti-angiogenesis ability. In 1998, Castellino et al. replaced the Lys binding site on kringle 5 domain, Leu-71, with Arg, resulting in the stronger affinity of kringle 5 with Lys (Chang Y, Mochalkin I, McCance S G, et al. Structure and ligand binding determinants of the recombinant kringle 5 domain of human plasminogen. Biochemistry 1998; 37: 3258-3271). In a preferred embodiment of the invention, it was shown that triple mutations (N289A/T346A/L532R) enhanced the anti-angiogenic ability of K₁₋₅. The glycosylation of plasminogen might mask its Lys binding site, and affect its Lys binding ability. It was also demonstrated that increased Lys binding ability of kringle 5 domain plus elimination of glycosylation sites at Asn-289 and Thr-346 enhances the interaction between K₁₋₅ with its ligand, integrin αᵥβ₃. It was demonstrated that the binding of angiostatin to αᵥβ₃ is related to angiostatin's Lys binding capacity. The result that the mutant with enhanced Lys binding capacity increased the interaction of K₁₋₅ with ° vp3 is consistent with the previous finding (Tarui T, Miles L A, Takada Y Specific interaction of angiostatin with integrin alpha(v)beta(3) in endothelial cells. J Biol Chem 2001; 276: 39562-39568; Tarui T, Akakura N, Majumdar M, et al. Direct interaction of the kringle domain of urokinase-type plasminogen activator (uPA) and integrin alpha v beta 3 induces signal transduction and enhances plasminogen activation. Thromb Haemost 2006; 95: 524-34) and this effect was not mediated by integrin α₂β₁. Several prior studies also suggested that angiostatin-related proteins inhibit endothelial cell function by activating focal-adhesion kinase and inducing apoptosis of endothelial cells (Veitonmaki N, Cao R, Wu L H, et al. Endothelial cell surface ATP synthase-triggered caspase-apoptotic pathway is essential for k1-5-induced antiangiogenesis. Cancer Res 2004; 64: 3679-3686). The apoptosis percentage of endothelial cells was measured after incubating with 200 nM wild-type K₁₋₅ or mutant K₁₋₅ proteins. It was demonstrated that mutated K₁₋₅ at residue Asn-289, Thr-346, and Leu-532 increased the ability to induce endothelial cell apoptosis. The result was consistent with the observation that Lys binding might be involved in binding K₁₋₅ to αᵥβ₃ (Tarui T, Miles L A, Takada Y Specific interaction of angiostatin with integrin alpha(v)beta(3) in endothelial cells. Biol Chem 2001; 276: 39562-39568) and that increased Lys binding affinity has a profound effect on inhibiting proliferation and inducing apoptosis in endothelial cells.

Previous study results showed that systemic treatment of tumor-bearing mice with angiostatin prevents tumor growth and metastasis (O'Reilly M S, Holmgren L, Shing Y, et al. Angiostatin: a novel angiogenesis inhibitor that mediates the suppression of metastases by a Lewis lung carcinoma. Cell 1994; 79: 315-328; O'Reilly M S, Holmgren L, Chen C, et al. Angiostatin induces and sustains dormancy of human primary tumors in mice. Nat Med 1996; 2: 689-692).

In this invention, it is demonstrated that replacement of the glycosylation site at residue Asn-289 or/and Thr-346 by Ala or/and enhanced Lys binding property will enhance the anti-angiogenic and anti-tumor actions of K₁₋₅, possibly through increased interaction with integrin αᵥβ₃ in endothelial cells. The increase of Lys binding ability and the altered glycosylation sites of mutant K₁₋₅ proteins may provide a new strategy in improving the anti-angiogenic function of K₁₋₅.

While the description sets forth various embodiment specific details, it will be appreciated that the description is illustrative only and should not to be construed in any way as limiting the invention. Furthermore, various applications of the invention, and modifications thereto, which may occur to those who are skilled in the art, are also encompassed by the general concepts described below.

The present invention relates to an anti-angiogenic protein comprising a mutant kringle 1-5 (K₁₅) fragment of plasminogen, wherein the mutation has substitution of amino acid residue of SEQ ID No. 6 at amino acid position selected from the group consisting of 227, 284 and 470, and wherein the position 227 is replaced with an amino acid residue without forming glycosylation, position 284 is replaced with an amino acid residue without forming glycosylation, and the position 470 is replaced with Arg or Leu; provided that the position 227 being Asn, position 284 being Thr and position 470 being Leu is excluded. SEQ ID NO. 6 is a polypeptide sequence containing plasminogen kringle 1-5 (K1-5) fragments. In wildtype K1-5, the position 227 is Asn-289, the position 284 is Thr-346 and the position 470 is Leu-532.

Said amino acid residue without forming glycosylation is a natural amino acid or an artificial amino acid. In one embodiment, the amino acid residue without forming glycosylation is selected from the group consisting of Ala, Ile, Leu, Met, Phe, Trp, Tyr, Val, Gln, Cys, Gly, Pro, Arg, His, Lys, Asp, Glu, Asn, Thr and their modified residues. More preferably, the amino acid residue without forming glycosylation is Ala.

The polypeptide sequence of the mutant kringle 1-5 fragment is preferably selected from the group consisting of SEQ ID NOs. 7, 8, 9, 10, 11, 12 and 13, more preferably SEQ ID NO. 13.

The anti-angiogenic protein can be used as a therapeutic agent in cancer therapy.

Said anti-angiogenic protein can inhibit angiogenesis in vivo, in vitro, in tumor tissue, or even exhibit anti-tumor effect. The effects of the anti-angiogenic protein are: (1) inhibition of endothelial cell proliferation; (2) induction of endothelial cell apoptosis; and (3) inhibition of endothelial cell migration.

In a preferred embodiment, the anti-angiogenic protein functions through a caspase-apoptotic pathway.

The anti-angiogenic protein can bind to an angiostatin receptor selected from the group consisting of angiomotin, endothelial cell surface ATP synthase, integrin, annexin II, C-met receptor, NG2-proteoglycans, tissue-type plasminogen activator, chondroitin sulfate proteoglycans, and CD26. In a preferred embodiment, the angiostatin receptor is integrin.

A nucleic acid having a sequence encoding said anti-angiogenic protein is also disclosed in the invention. The sequence is preferably selected from the group consisting of SEQ ID NOs. 16, 18, 20, 22, 24, 26 and 28, more preferably SEQ ID NO. 28. Said nucleic acid can be applied to cancer therapy.

The invention further relates to a pharmaceutical composition for inhibiting angiogenesis comprising said anti-angiogenic protein or said nucleic acid. In a preferred embodiment, the composition further comprises a pharmaceutically acceptable excipient, carrier or diluent. The pharmaceutically acceptable excipient, carrier or diluent may be adapted for oral, sublingual, rectal, nasal or parenteral administration. In a more preferred embodiment, the pharmaceutical composition can be applied to cancer therapy.

Also disclosed is a method for treatment of an angiogenesis associated disease or disorder, comprising administering to a patient (preferably a mammal) in need of such treatment an effective amount of said pharmaceutical composition. In a preferred embodiment, said method further comprises adding a pharmaceutically acceptable excipient, carrier or diluent. In a more preferred embodiment, the pharmaceutically acceptable excipient, carrier or diluent is adapted for oral, sublingual, rectal, nasal or parenteral administration.

The angiogenesis associated disease or disorder is preferably tumor metastasis, diabetic retinopathy, sickle cell anemia, vein occlusion, artery occlusion, macular degeneration, atherosclerosis, rheumatoid arthritis, systemic lupus, osteoarthritis, obesity, psoriasis or restenosis. In a more preferred embodiment, said method for treatment of an angiogenesis associated disease or disorder can be applied to cancer therapy.

### EXAMPLES

The examples below are non-limiting and are merely representative of various aspects and features of the present invention.

### Example 1

### Experimental Procedures

### Materials

Human Glu-plasminogen was isolated from human plasma as described previously (16). EasySelect Pichia Expression Kit, Dulbecco modified Eagle medium (DMEM), fetal bovine serum (FBS), and trypsin solution were purchased from Invitrogen (Carlsbad, Calif., USA). All restriction enzymes were from New England Biolabs, Inc. (Beverly, Mass., USA). Anti-plasminogen polyclonal antiserum from mice was prepared in our laboratory. The antibodies against integrin αᵥβ₃ (clone: LM609) and integrin α₂β₁ (clone: BHA2.1) were from United Chemicon (Rosemont, Ill., USA). Anti-angiostatin and anti-CD31 monoclonal antibodies were from BD Biosciences (Bedford, Mass., USA). Normal mouse IgG was from Santa Cruz Biotechnology, Inc. (Santa Cruz, Calif., USA). Sulfosuccinimidyl-2-[biotinamido]ethyl-1,3-dithiopropionate (Sulfo-NHS—SS-biotin) and BCA protein assay reagent kit was from Pierce (Rockford, Ill., USA). Basic fibroblast growth factor (bFGF) was from R&D Systems (Minneapolis, Minn., USA).

### Construction of Wild-Type and Mutant K₁₋₅

A cDNA encoding K₁₋₅ domain of plasminogen was amplified by PCR using a human liver cDNA library as the template with the oligonucleotide primers 5′-GGTACCGGTACCAAAGTGTATCTCTCAGAGTGC-3′ (SEQ ID NO.1) and 5′-GGGGTACCCCTTAGGCCGCACACTGAGGGACATCAC-3′ (SEQ ID NO.2), which contained linkers with Kpn I restriction sites. The amplified 1398 bps fragment, spanning from residue 78 to 543 of human plasminogen, was digested with Kpn I and cloned into pre-digested P. pastoris expression vector pPICZαA, which contained an alcohol oxidase gene and a secretion signal for secreting target recombinant proteins to make pPICZαA/K₁₋₅. The site-directed mutagenesis was performed by the PCR-based method following the manufacturer's instructions (Quick Change Site-directed Mutagenesis Kit, Stratagene, Boston) as described previously. Pairs of specific primers were synthesized to create codons for Ala at Asn-289 or Thr-346, and for Arg at Leu-532. The following primers were used (only mutagenic forward primers are shown):

| | |
|---|---|
| Asn289Ala | |
| forward: | |
| 5′-ACACACATGCGCGCACACCAG-3′ | (SEQ ID NO. 3) |
| Thr346Ala | |
| forward: | |
| 5′-AATTGGCTCCTGCAGCACCACCTG-3′ | (SEQ ID NO. 4) |
| Leu532Arg | |
| forward: | |
| 5′-ATCCAAGAAAACGTTACGACTACT-3′ | (SEQ ID NO. 5) |

The fidelity of the constructs was confirmed by sequencing. The altered cDNA was fully sequenced and subcloned into pPICZαA expression vector.

### Expression and Purification of Wild-Type and Mutant K₁₋₅

X-33 cells were transformed with wild-type K₁₋₅ and mutant construct K₁₋₅ linearized with Sac I by the Pichia EasyComp™ Kit (Invitrogen Corp). Cells were plated onto YPD agar (yeast extract peptone dextrose medium: 1% yeast extract, 2% peptone, and 2% dextrose) containing 1 mg/ml zeocin. Resultant clones were screened for expression. Large-scale expression of kringle proteins was performed in a 5-liter fermenter (B. Braun Biotech). Crude culture broth containing recombinant K₁₋₅ proteins was clarified by centrifugation at 14,000 g for 20 minutes and was dialyzed against 100 mM phosphate buffer (pH 7.0). After dialysis, culture broth was applied at 1 ml/min to DEAE-Sepharose column previously equilibrated with 100 mM sodium phosphate, pH 7.0. The flow-through was then loaded into a column containing 10 ml Lys-Sepharose 4B (Amersham Pharmacia Biotech), previously equilibrated with binding buffer (100 mM phosphate buffer, pH 7.0). The column was washed with 10 volumes of binding buffer, and eluted with 0.2 M EACA (Sigma-Aldrich) to recover wild-type and mutant K₁₋₅ proteins. The preparations were subjected to Western blot analysis, and the purity was evaluated to be >95% by Coomassie blue of SDS-PAGE.

### Preparation of Proteolytic Fragments of Native Human K₁₋₅

Approximately 40 mg of purified Glu¹-plasminogen in 4 ml of 0.1 M glycine buffer (pH 10.5) was incubated at 25° C. with immobilized urokinase-activated plasmin for 14 hours. After incubation, the sample was applied to a Lys-Sepharose column (1.0×30 cm) pre-equilibrated and washed with 1.0 M sodium phosphate buffer (pH 8.0). A peak containing micro-plasminogen was detected in the flow-through fraction. K₁₋₅ was eluted from the column with a 0-1 M linear gradient of EACA. The protein fraction containing K₁₋₅ was further purified with a Sephadex G-75 column (2.6×90 cm) (17). The purified K₁₋₅ was dialyzed against distilled H₂O and lyophilized. The purity of native K₁₋₅ was analyzed by SDS-PAGE.

### SDS-Polyacrylamide Gel Electrophoresis

SDS-PAGE was performed according to the procedure of Laemmli using 12.5% separating gel under a reduced condition (18).

### Protein Concentration

The protein concentration of recombinant proteins was measured with the BCA kit using bovine serum albumin as a standard.

### Sequence Analysis

The aminoacid sequence determinations were carried out by Edman degradation in an Applied Biosystems Sequencer (model 477A).

### Cell Culture

Human microvascular endothelial cells (HMECS) and bovine aortic endothelial cells (BAECS) were obtained from Cambrex Corporation (East Rutherford, N.J., USA) and cultured in Endothelial Cell Basal Medium-2 (EBM-2) and DMEM as recommended by the supplier. In all experiments, cells were used between the fifth and eighth passages.

### Endothelial Cell Proliferation Assay

BAECs and HMECs (3000 cells) were added to 96-well plates and incubated at 37° C. for 6 h. The medium was replaced with 0.2 ml of fresh DMEM containing 2% FBS, 10 ng/ml bFGF, and kringle proteins in triplicates. To evaluate the effect of kringle proteins on DNA synthesis in endothelial cells, we performed a 5-bromo-2′-deoxyuridine (BrdU) incorporation assay using a commercial quantification kit (Roche Diagnostic GmbH, Mannheim, Germany) and following manufacturer's protocol. The absorbance at 450 nm was measured with an Enzyme-Linked ImmunoSorbent Assay (ELISA) reader (Molecular Device, Sunnyvale, Calif., USA).

### Endothelial Cell Apoptosis Detection

BAECs and HMECs (4×10⁵ cells) were cultured and incubated at 37° C. in 10% CO₂ for 16 h. The medium was replaced with 2 ml of fresh DMEM containing 2% FBS, and 200 nM wild-type or mutant K₁₋₅ proteins with 10 ng/ml bFGF. After 24 h, cells were trypsinized, washed with phosphate-buffered saline (PBS), and then fixed in 70% ethanol. After fixation, cells were collected by centrifugation at 2000 rpm for 5 min. Finally 500 μl PBS and 30 μl propidium iodide (20 μg/ml) were added. Cells were assessed by flow cytometry (FACSort, BD Biosciences) and the results were analyzed with CellQuest software.

### In Vivo Angiogenesis Assay

Matrigel plug assay was performed to examine the anti-angiogenesis effect of wild-type and mutant K₁₋₅ proteins in vivo. A potent angiogenic mixture of bFGF (300 ng/ml) was added to Matrigel containing 60 U/ml heparin pre-incubated at 4° C. in the absence or presence of various concentrations of kringle proteins (wild-type K₁₋₅, K₁₋₅N289A, K₁₋₅T346A, K₁₋₅L532R, K₁₋₅N289A/T346A/L532R), to a final volume of 500 μl. The Matrigel suspension was slowly injected subcutaneously into the flanks of C57BL6/J mice using a cold syringe. After 7 days, the mice were sacrificed and the Matrigel plugs were surgically removed. Gels were collected, weighed, and subjected to analysis of hemoglobin content as an estimate of vascularization as described previously. Hemoglobin was measured using the Drabkin method and Drabkin reagent kit 525 (Sigma-Aldrich). The concentration of hemoglobin was calculated from a known amount of hemoglobin assayed in parallel. For histological analysis, the recovered Matrigel plugs were immediately frozen in liquid nitrogen. Sections were made at 5 μm thick and post-fixed for 5 minutes in −20° C. methanol. Immunostaining of CD31 was then performed. The sections were blocked with 10% normal goat serum (Sigma-Aldrich) for 30 minutes, stained with rat anti-mouse CD31 (PECAM-1, platelet endothelial cell adhesion molecule-1) antibody in a 1:100 dilution, and then followed by Alexa Fluor 546 goat anti-rat antibody (Molecular Probes Inc.) in a 1:200 dilution. Specimens were examined and photographed using a Leica DMLB microscope and Leica DC 180 digital camera.

### Adhesion Assays

Adhesion assays were performed as previously described (20). Briefly, the 96-well Immulon-2 microtiter plates (Dynatech Laboratories, Chantilly, Va., USA) were coated with 100 μl of PBS containing BSA or K₁₋₅ proteins at a concentration of 15-250 nM and incubated for 1 h at 37° C. Equivalent amounts of each K₁₋₅ protein with the same concentration were bound to the plate as checked by ELISA assay to eliminate the possibility that one protein may bind to the plate better than the other. The remaining protein binding sites were blocked by incubating with 0.2% BSA for 1 h at room temperature. Cells (10⁵ cells) in 1001 of Hepes-Tyrode buffer supplemented with 2 mM MgCl₂ in triplicate were added to the wells and incubated at 37° C. for 1 h. After non-bound cells were removed by rinsing the wells with the same buffer, bound cells were quantified by measuring endogenous phosphatase activity. The negative control was lysis/substrate solution only without BAECs and the absorbance was about 0.1482. The positive control was detected with 1×10⁵ cells whole-cell lysate and its absorbance was about 0.398.

### Tumor Study in Mice

Animal experiments were performed to evaluate the suppression efficiency of wild-type K₁₋₅ and K₁₋₅N289A/T346A/L532R on angiogenesis-dependent tumor growth. Male 6-week-old C57BL6/J mice were used for tumor studies. Approximately 1×10⁶ Lewis lung carcinoma cells growing in logarithmic phase were harvested and resuspended in PBS, and a single cell solution of 200 pt was implanted subcutaneously in the middle dorsum of each animal. After 7 days, when tumors became palpable, tumor-bearing mice were subcutaneously injected with either 100 μl of PBS or 100 μl of wild-type K₁₋₅ and K₁₋₅N289A/T346A/L532R (2.5 mg/kg/day) in PBS once per two days for 21 days. Primary tumors were measured with digital calipers on the days indicated. Tumor volumes were calculated according to the formula: width²×length×0.52 as reported (19). The mice were sacrificed at day 21 and the tumor masses were surgically removed. The recovered tumor masses were immediately frozen in liquid nitrogen. Sections were made at 5 μm thick and post-fixed for 5 minutes in −20° C. methanol. Immunostaining of CD31 was then performed as described previously.

### ELISA

BAECs (3000 cells) were added to 96-well plates and incubated at 37° C. for 6 h. The biotin-labeled kringle proteins were added to each well. After 60 min at 37° C. without shaking, the plate wells were washed once with wash buffer, 1001 of streptavidin peroxidase was added and incubation continued for 20 min. Then the plate wells were washed twice with wash buffer, and 1001 tetramethyl benzidine was added and incubated for 10 min. The reaction was stopped with 50 μl of 2NH₂SO₄. Absorbance of each well was then read at 450 nm.

### Animal Care

Animal experiments were approved by the Institutional Animal Care and Use Committee of National Cheng Kung University, Tainan, Taiwan.

### Statistical Analysis

Statistical analysis was carried out using the unpaired Student t test. Differences between >2 groups were compared by One-way ANOVA in GraphPad Prism. A value of p<0.05 was considered to be statistically significant.

### Example 2

### Purification and Characterization of K₁₋₅ Proteins

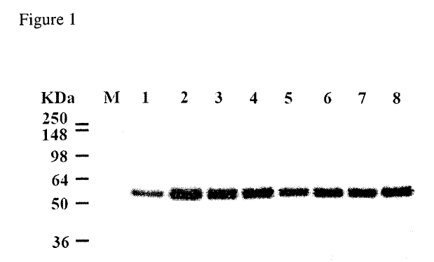

To obtain wild-type K₁₋₅ protein (SEQ ID NO. 6, wherein the position 227 is Asn-289, the position 284 is Thr-346, and the position 470 is Leu-532) and mutant K₁₋₅ proteins (K₁₋₅N289A, K₁₋₅T346A, K₁₋₅L532R, K₁₋₅N289A/T346A, K₁₋₅T346A/L532R, K₁₋₅N289A/L532R, and K₁₋₅N289A/T346A/L532R, as in SEQ ID NOs. 7, 8, 9, 10, 11, 12 and 13) in a soluble form, the P. pastoris expression system was employed by using nucleic acid sequences encoding them. The nucleic acid sequences encoding wild-type K₁₋₅ and mutant K₁₋₅ proteins K₁₋₅N289A, K₁₋₅T346A, K₁₋₅L532R, K₁₋₅N289A/T346A, K₁₋₅T346A/L532R, K₁₋₅N289A/L532R, and K₁₋₅N289A/T346A/L532R were SEQ ID NOs. 14, 16, 18, 20, 22, 24, 26 and 28, respectively. Purified wild-type and mutant K₁₋₅ proteins had one major protein band with molecular mass of 56 kDa (FIG. 1), and these proteins could be recognized by anti-angiostatin monoclonal antibody and anti-plasminogen polyclonal antiserum. The NH₂-terminal amino acid sequencing results showed that these proteins had an expected single sequence starting at the fusion peptide derived from the Ste13 site, followed by the sequence of human plasminogen Lys-78. In addition, the Lys binding features of wild-type K₁₋₅ and K₁₋₅N289A/T346A/L532R were detected by immobilized Lys-Sepharose gels. The concentration of EACA to elute the wild-type K₁₋₅ and K₁₋₅N289A/T346A/L532R from Lys-Sepharose gels was 42 mM for K₁₅ and 54 mM for K₁₅N289A/T346A/L532R. This result demonstrated that the Lys binding ability of K₁₋₅N289A/T346A/L532R was increased.

### Example 3

### Dose-Dependent Inhibition of Endothelial Cell Proliferation by K₁₋₅ Proteins

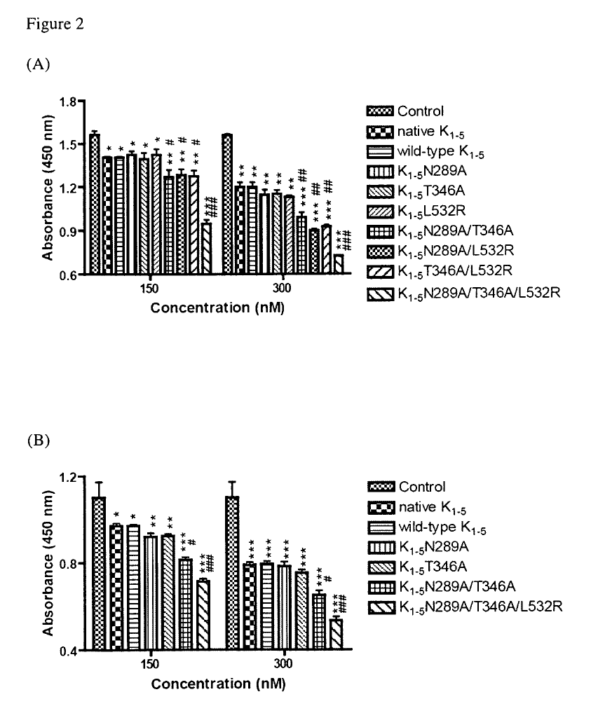

To evaluate the anti-angiogenesis ability of wild-type and mutant K₁₋₅ proteins, purified proteins were assayed for their effect on the proliferation of BAECs and HMECs. As shown in FIG. 2A, wild-type K₁₋₅ inhibited bFGF-stimulated BAECs growth as effective as native K₁₋₅. Mutant K₁₋₅ proteins also exhibited inhibitory effect on bFGF-stimulated endothelial cell proliferation. The result showed that lack of glycosylation site and increased Lys binding ability mutants, K₁₋₅N289A/T346A, K₁₋₅N289A/L532R and K₁₋₅T346A/L532R, had higher inhibitory activity than the wild-type K₁₋₅ (FIG. 2A). Among these mutant K₁₋₅ proteins, the inhibitory ability of K₁₋₅N289A/T346A/L532R was the greatest. Similar result was observed in HMECs (FIG. 2B). This indicates that the alteration of glycosylation sites and Lys binding ability within K₁₋₅ may enhance the inhibitory ability in bFGF-stimulated endothelial cell proliferation.

### Example 4

### Endothelial Cell Apoptosis Detection

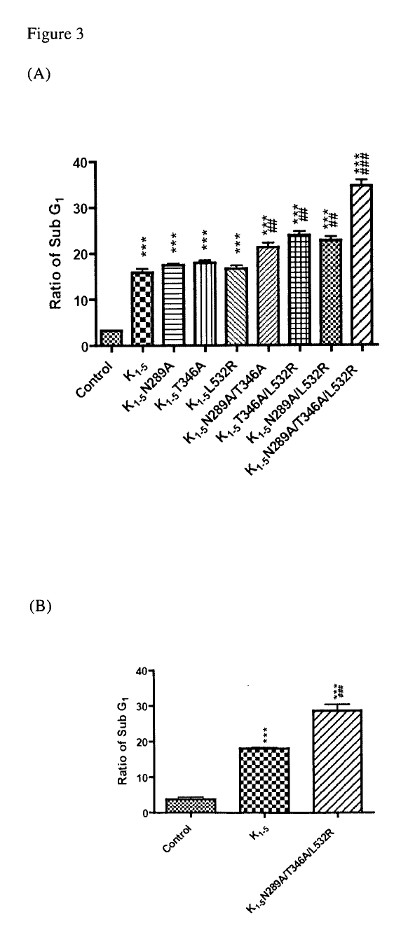

To determine the cytotoxic effect of wild-type and mutants K₁₋₅ proteins, BAECs were treated with 200 nM wild-type or mutants K₁₋₅ proteins and bFGE After 24 h, cells that had undergone apoptosis were stained by propidium iodide and detected by flow cytometry. As shown in FIG. 3A, the ratio of sub G₁ in untreated cells was 3.27%. The treatment of wild-type or mutant K₁₋₅ proteins induced different degrees of apoptosis. Compared with 15.88% in the treatment of wild-type K₁₋₅, about 34.58% of cells underwent apoptosis with the treatment of K₁₋₅N289A/T346A/L532R. Similar effect was also observed in HMECs (FIG. 3B). Therefore, mutation at these residues enhanced the ability of K₁₋₅ to induce endothelial cell apoptosis.

### Example 5

### In Vivo Angiogenesis Assay

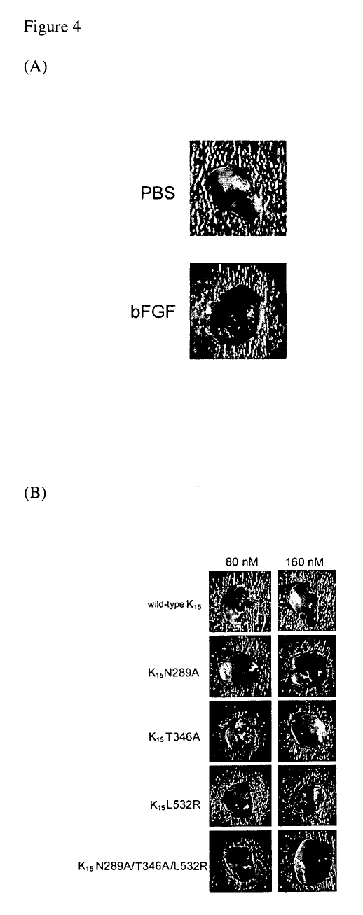

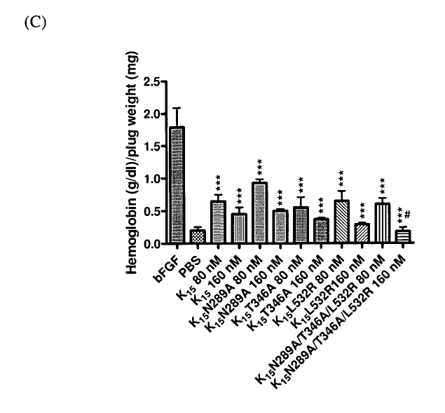

To test the anti-angiogenic action of wild-type and mutant K₁₋₅ proteins in vivo, Matrigel implant models were performed. At first, the effect of bFGF-induced angiogenesis was tested and the potency of angiogenesis induced by bFGF reached maximum at day 7. As shown in FIG. 4A, the negative control showed a clear color compared to that containing bFGF, which showed bright red color. The results showed that a strong angiogenesis effect was induced in the gel containing bFGF. In contrast, neovasculization was inhibited in gels containing wild-type K₁₋₅ or mutant K₁₋₅ proteins with concentrations of both 80 and 160 nM (FIG. 4B). Furthermore, the hemoglobin content in these gels was measured as an indicator of angiogenic response. The result showed that the mean hemoglobin level was significantly lower in gels treated with K₁₋₅ proteins (wild-type K₁₋₅, K₁₋₅N289A, K₁₋₅T346A, K₁₋₅L532R, and K₁₋₅N289A/T346A/L532R) than in gels with bFGF only (FIG. 4C). The 80 nM concentration of wild-type K₁₋₅ and mutant K₁₋₅ had similar potency in inhibiting angiogenesis, as shown in FIG. 4C. However, at 160 nM concentration, K₁₋₅N289A/T346A/L532R showed the greatest inhibitory effect. The results showed that these K₁₋₅ proteins were most potent in inhibiting bFGF-induced angiogenesis in vivo.

### Example 6

### Suppression of Primary Tumor Growth by Systemic Administration of K₁₋₅ Proteins

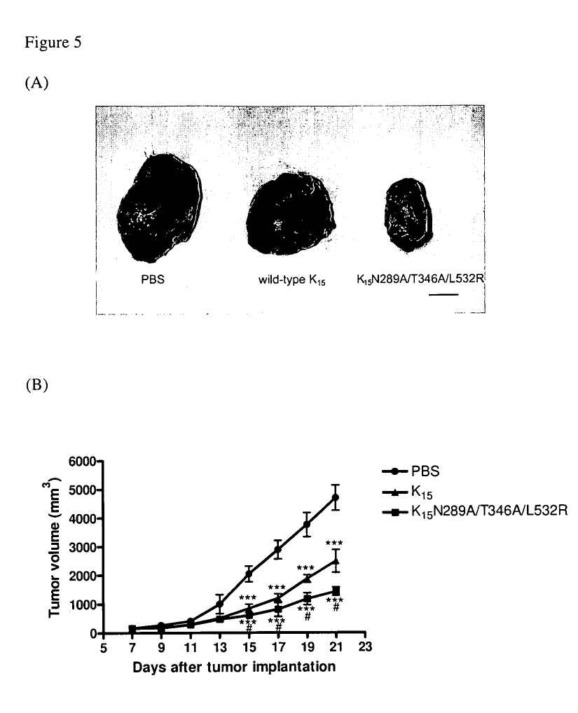

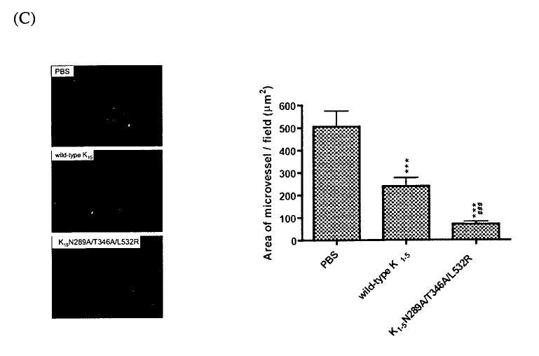

Angiogenesis is known to be activated during the early stages of tumor development, and angiogenesis inhibitors have different degrees of efficiency depending on the stage of carcinogenesis. Treatment with wild-type K₁₋₅ and K₁₋₅N289A/T346A/L532R resulted in a significant suppression of primary tumor growth as shown in FIGS. 5A and 5B. The ratio of mean tumor volume of treated mice over control mice (T/C) was 0.52 and 0.30 for wild-type K₁₋₅ and K₁₋₅N289A/T346A/L532R. The results showed that K₁₋₅N289A/T346A/L532R was more effective in inhibiting primary tumor growth in vivo. In order to determine the anti-angiogenic effect, tissues of primary tumor were further stained by anti-CD31 antibody. As shown in FIG. 5C, tissues of control mice were highly stained by anti-CD31 antibody. However, the treatment of wild-type K₁₋₅ and K₁₋₅N289A/T346A/L532R significantly reduced new blood vessel formation in the primary tumor (FIG. 5C). The inhibitory effect of tumor growth by wild-type K₁₋₅ and K₁₋₅N289A/T346A/L532R is the result of inhibition of endothelial-specific angiogenesis.

### Example 7

### Specific Interaction of K₁₅ Proteins with Integrin αᵥβ₃ in Endothelial Cells

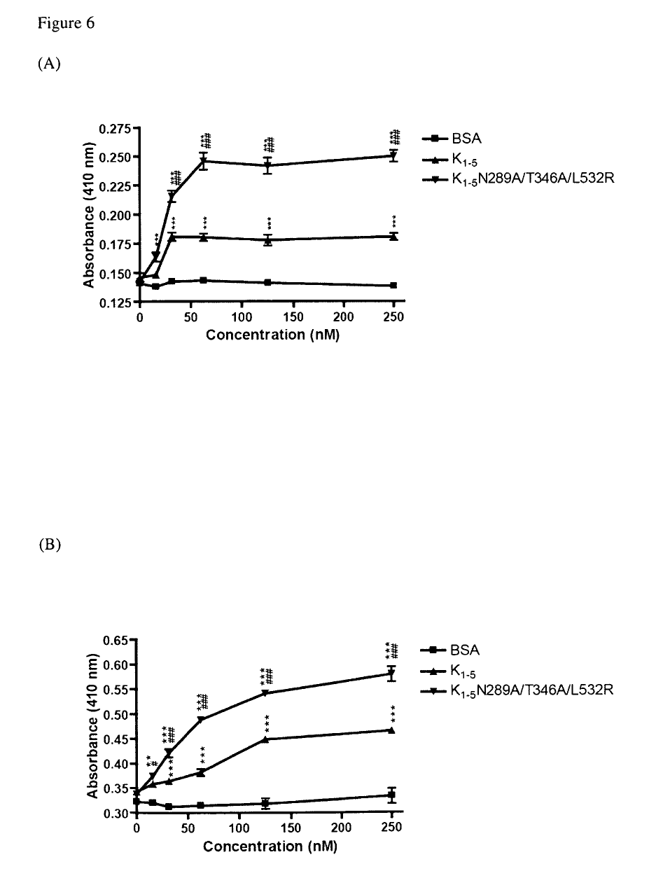

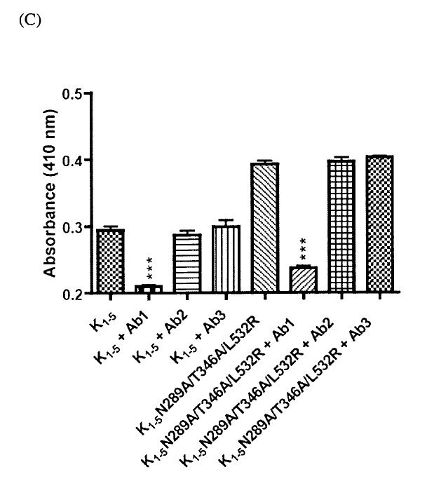

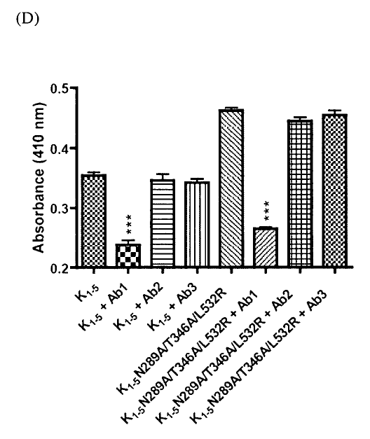

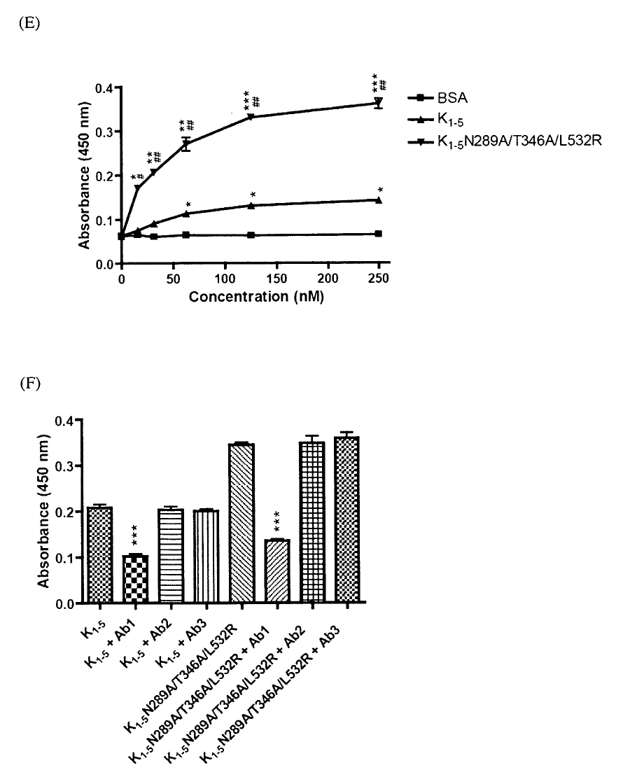

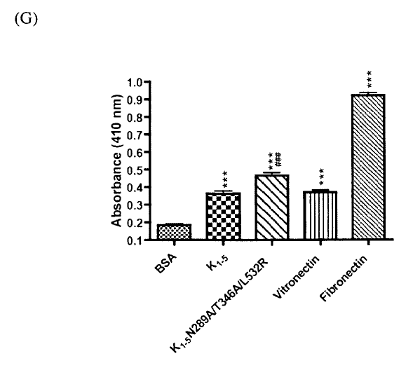

To determine whether integrin αᵥβ₃ was involved in the binding of K₁₋₅ to BAECs and HMECs, the adhesion assay was performed. As shown in FIGS. 6A and 6B, both cell types could adhere to K₁₋₅-coated wells and the ability of both cells to adhere to K₁₋₅N289A/T346A/L532R was greater than K₁₋₅. This result indicated that altering glycosylation sites and Lys binding properties of K₁₋₅ may enhance the ability of K₁₋₅ to bind to endothelial cells. To understand the role of integrin αᵥβ₃ in cells binding to K₁₋₅ proteins, antibody against integrin αᵥβ₃ or integrin α₂β₁ was used. As shown in FIGS. 6C and 6D, the ability of endothelial cells to bind to K₁₋₅ or K₁₋₅N289A/T346A/L532R was inhibited by 10 μg/ml anti-integrin αᵥβ₃ antibody, suggesting that integrin αᵥβ₃ mediated the adhesion of K₁₋₅ to endothelial cells. In addition, K₁₋₅ and K₁₋₅N289A/T346A/L532R were labeled with biotin to evaluate the binding of kringle proteins to endothelial cells. As shown in FIGS. 6E and 6F, the binding ability of K₁₋₅N289A/T346A/L532R to BAECs was greater than K₁₋₅ and this effect could be inhibited by anti-integrin αᵥβ₃, but not by anti-integrin α₂β₁ antibody treatment. The adhesion capacity of HMECs to kringle proteins was similar to that of vitronectin, but lower than that of fibronectin (FIG. 6G).

### Example 8

### K₁₋₅ Proteins Inhibited Endothelial Cell Proliferation Through Integrin αᵥβ₃

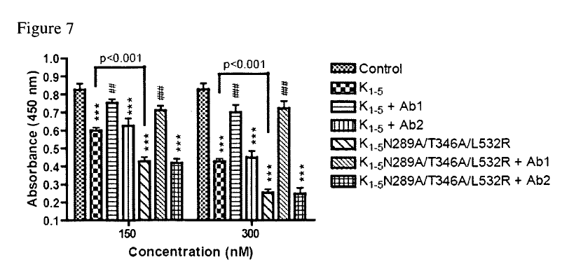

According to the observation presenting in FIG. 6, we demonstrated that integrin αᵥβ₃ might play a pivotal role in the anti-angiogenesis effect of kringle proteins. To further investigate the role of integrin αᵥβ₃ on the anti-proliferative effect of kringle proteins, HMECs were pretreated with 10 μg/ml anti-integrin αᵥβ₃ or anti-integrin α₂β₁ antibody for 30 min and then treated with kringle proteins for 3 days. The result showed that anti-integrin αᵥβ₃ antibody partially reversed the inhibitory effect of kringle proteins on endothelial cell proliferation, while anti-integrin α₂β₁ antibody had no such effect (FIG. 7).

One skilled in the art readily appreciates that the present invention is well adapted to carry out the objects and obtain the ends and advantages mentioned, as well as those inherent therein. The cell lines, embryos, animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

What is claimed is:

1. An anti-angiogenic protein comprising a mutant kringle 1-5 (K₁₅) fragment of plasminogen, wherein the mutation has substitution of amino acid residue of SEQ ID NO. 6 at amino acid position selected from the group consisting of 227, 284 and 470, and wherein the position 227 is replaced with an amino acid residue without forming glycosylation, position 284 is replaced with an amino acid residue without forming glycosylation, and the position 470 is replaced with Arg or Leu; provided that the position 227 being Asn, position 284 being Thr and position 470 being Leu is excluded.

2. The anti-angiogenic protein of claim 1, wherein the amino acid residue without forming glycosylation is a natural amino acid or an artificial amino acid.

3. The anti-angiogenic protein of claim 1, wherein the amino acid residue without forming glycosylation is selected from the group consisting of Ala, Ile, Leu, Met, Phe, Trp, Tyr, Val, Gln, Cys, Gly, Pro, Arg, His, Lys, Asp, Glu, Asn, Thr and their modified residues.

4. The anti-angiogenic protein of claim 1, wherein polypeptide sequence of the mutant kringle 1-5 fragment is selected from the group consisting of SEQ ID NOs. 7, 8, 9, 10, 11, 12 and 13.

5. The anti-angiogenic protein of claim 4, wherein polypeptide sequence of the mutant kringle 1-5 fragment is SEQ ID NO. 13.

6. The anti-angiogenic protein of claim 1, which inhibits angiogenesis in vivo and in vitro.

7. The anti-angiogenic protein of claim 1, which inhibits angiogenesis in tumor tissue.

8. The anti-angiogenic protein of claim 1, which exhibits anti-tumor effect.

9. The anti-angiogenic protein of claim 1, which inhibits endothelial cell proliferation and/or induces endothelial cell apoptosis.

10. The anti-angiogenic protein of claim 1, which inhibits endothelial cell migration.

11. The anti-angiogenic protein of claim 1, which inhibits Akt and/or eNOS phosphorylation.

12. The anti-angiogenic protein of claim 1, which functions through a caspase-apoptotic pathway.

13. The anti-angiogenic protein of claim 1, which binds to an angiostatin receptor selected from the group consisting of angiomotin, endothelial cell surface ATP synthase, integrin, annexin II, C-met receptor, NG2-proteoglycans, tissue-type plasminogen activator, chondroitin sulfate proteoglycans, and CD26.

14. The anti-angiogenic protein of claim 13, wherein the angiostatin receptor is integrin.

15. The anti-angiogenic protein of claim 1, which is a therapeutic agent in cancer therapy.

16. A nucleic acid having a sequence encoding said anti-angiogenic protein of claim 1.

17. The nucleic acid of claim 16, wherein nucleic acid sequence of the mutant kringle 1-5 fragment is selected from the group consisting of SEQ ID NOs. 16, 18, 20, 22, 24, 26 and 28.

18. The nucleic acid of claim 17, wherein nucleic acid sequence of the mutant kringle 1-5 fragment is SEQ ID NO. 28.

19. The nucleic acid of claim 16, which can be applied to cancer therapy.

20. A pharmaceutical composition comprising said anti-angiogenic protein of claim 1 or said nucleic acid of claim 16.

21. The pharmaceutical composition of claim 20, which further comprises a pharmaceutically acceptable excipient, carrier or diluent.

22. A method for treatment of an angiogenesis associated disease or disorder, comprising administering to a patient in need of such treatment an effective amount of said pharmaceutical composition of claim 20.

23. The method of claim 22, wherein the angiogenesis associated disease or disorder is tumor metastasis, diabetic retinopathy, sickle cell anemia, vein occlusion, artery occlusion, macular degeneration, atherosclerosis, rheumatoid arthritis, systemic lupus, osteoarthritis, obesity, psoriasis or restenosis.

24. The method of claim 22, which can be applied to cancer therapy.

25. The method of claim 23, wherein the patient is mammal.


SEQUENCE LISTING (29 sequences)

SEQ ID NO: 1 - DNA, 33 bp, Artificial
  An oligonucleotide primer, which is used to amplify a cDNA encoding K15 domain of plasminogen from human liver cDNA library.
ggtaccggtaccaaagtgtatctctcagagtgc

SEQ ID NO: 2 - DNA, 36 bp, Artificial
  An oligonucleotide primer, which is used to amplify a cDNA encoding K15 domain of plasminogen from human liver cDNA library.
ggggtaccccttaggccgcacactgagggacatcac

SEQ ID NO: 3 - DNA, 21 bp, Artificial
  An oligonucleotide primer for site-directed mutagenesis, which is used to create codons for Ala at Asn-289 of human kringle 1-5.
acacacatgcgcgcacaccag

SEQ ID NO: 4 - DNA, 24 bp, Artificial
  An oligonucleotide primer for site-directed mutagenesis, which is used to create codons for Ala at Thr-346 of human kringle 1-5.
aattggctcctgcagcaccacctg

SEQ ID NO: 5 - DNA, 24 bp, Artificial
  An oligonucleotide primer for site-directed mutagenesis, which is used to create codons for Arg at Leu-532 of human kringle 1-5.
atccaagaaaacgttacgactact

SEQ ID NO: 6 - protein, 481 aa, Artificial
  A polypeptide sequence of plasminogen K15. In wildtype K15, the position 227 is Asn-289, the position 284 is Thr-346 and the position 470 is Leu-532.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHXRTPENFPCKNLDENYCRNPDGKRAPWCHTTNSQVRWEYCKIPSCDSSPVSTEQLAPXAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKXYDYCDVPQCAA

SEQ ID NO: 7 - protein, 481 aa, Artificial
  A polypeptide sequence of K15N289A.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHARTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 8 - protein, 481 aa, Artificial
  A polypeptide sequence of K15T346A.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPAAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 9 - protein, 481 aa, Artificial
  A polypeptide sequence of K15L532R.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKRYDYCDVPQCAA

SEQ ID NO: 10 - protein, 481 aa, Artificial
  A polypeptide sequence of K15N289A/T346A.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHARTPENFPCKNLDENYCRNPDGKRAPWCHTTNSQVRWEYCKIPSCDSSPVSTEQLAPAAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 11 - protein, 481 aa, Artificial
  A polypeptide sequence of K15T346A/L532R.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPAAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKRYDYCDVPQCAA

SEQ ID NO: 12 - protein, 481 aa, Artificial
  A polypeptide sequence of K15N289A/L532R.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHARTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKRYDYCDVPQCAA

SEQ ID NO: 13 - protein, 481 aa, Artificial
  A polypeptide sequence of K15N289A/T346A/L532R.
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHARTPENFPCKNLDENYCRNPDGKRAPWCHTTNSQVRWEYCKIPSCDSSPVSTEQLAPAAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKRYDYCDVPQCAA

SEQ ID NO: 14 - DNA, 1443 bp, Artificial
  A nucleic acid sequence of wildtype K15.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggcc

SEQ ID NO: 15 - protein, 481 aa, Artificial
  Synthetic Construct
EAEFTWPSRPSRIGTKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAA

SEQ ID NO: 16 - DNA, 1443 bp, Artificial
  A nucleic acid sequence of K15N289A.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacatgcgcgcacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggcc

SEQ ID NO: 17 - protein, 481 aa, Artificial; identical to SEQ ID NO: 7
  Synthetic Construct

SEQ ID NO: 18 - DNA, 1443 bp, Artificial
  A nucleic acid sequence of K15T346A.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcctgcagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggcc

SEQ ID NO: 19 - protein, 481 aa, Artificial; identical to SEQ ID NO: 8
  Synthetic Construct

SEQ ID NO: 20 - DNA, 1443 bp, Artificial
  A nucleic acid sequence of K15L532R.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaacgttacgactactgtgatgtccctcagtgtgcggcc

SEQ ID NO: 21 - protein, 481 aa, Artificial; identical to SEQ ID NO: 9
  Synthetic Construct

SEQ ID NO: 22 - DNA, 1443 bp, Artificial
  A nucleic acid sequence of K15N289A/T346A.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacatgcgcgcacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcctgcagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggcc

SEQ ID NO: 23 - protein, 481 aa, Artificial; identical to SEQ ID NO: 10
  Synthetic Construct

SEQ ID NO: 24 - DNA, 1443 bp, Artificial
  A nucleic acid sequence of K15T346A/L532R.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcctgcagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaacgttacgactactgtgatgtccctcagtgtgcggcc

SEQ ID NO: 25 - protein, 481 aa, Artificial; identical to SEQ ID NO: 11
  Synthetic Construct

SEQ ID NO: 26 - DNA, 1443 bp, Artificial
  A nucleic acid sequence of K15N289A/L532R.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacatgcgcgcacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaacgttacgactactgtgatgtccctcagtgtgcggcc

SEQ ID NO: 27 - protein, 481 aa, Artificial; identical to SEQ ID NO: 12
  Synthetic Construct

SEQ ID NO: 28 - DNA, 1443 bp, Artificial
  A nucleic acid sequence of K15N289A/T346A/L532R.
gaagctgaattcacgtggcccagccggccgtctcggatcggtaccaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgtgacatcccccgctgcacaacacctccaccatcttctggtcccacctaccagtgtctgaagggaacaggtgaaaactatcgcgggaatgtggctgttaccgtgtccgggcacacctgtcagcactggagtgcacagacccctcacacacatgcgcgcacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcctgcagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaacgttacgactactgtgatgtccctcagtgtgcggcc

SEQ ID NO: 29 - protein, 481 aa, Artificial; identical to SEQ ID NO: 13
  Synthetic Construct